(19) United States
(12) Patent Application Publication
(10) Pub. No.: US 2010/0317921 A1
(43) Pub. Date: Dec. 16, 2010

(54) BALLOON EXPANDABLE INTRAVASCULAR BASKET CATHETER

(75) Inventors: Eric T. Marple, Loxahatchee, FL (US); Nicholas Green, Juniper, FL (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/813,312

(22) Filed: Jun. 10, 2010

Related U.S. Application Data
(60) Provisional application No. 61/186,199, filed on Jun. 11, 2009.

(51) Int. Cl. A61B 1/00

(52) U.S. Cl. 600/116

(57) ABSTRACT

The invention provides balloon-expandable intravascular basket-style catheters optimized for efficient interrogation of blood vessel walls. Related diagnostic systems and methods are also provided.

### CROSS-REFERENCE TO RELATED APPLICATIONS

This application claims the benefit of U.S. provisional patent application Ser. No. 61/186,199 filed Jun. 11, 2009, which is incorporated by reference herein in its entirety.

### FIELD OF THE INVENTION

The invention relates to the field of diagnostic intravascular catheters and more specifically to basket-style intravascular catheters.

### BACKGROUND OF INVENTION

Existing “basket-style” multi-arm tissue contact catheters have been described in prior disclosures by the inventor and others, such as U.S. Pub. No. 2005/0107706, which is incorporated by reference herein in its entirety. These include various multi-arm tissue contact catheters, including various possible embodiments for deployment and retraction of the basket arms for easier delivery and compatibility with differently sized vessels.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety

U.S. Pat. No. 6,701,181 discloses multi-path optical catheters, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,949,072 discloses devices for vulnerable plaque detection, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

U.S. Publication No. 2004/00176699 discloses basket-type thermography catheters in which each probe arm is independently moveable, and is incorporated by reference herein in its entirety.

U.S. Publication No. 2004/0204651 discloses infrared endoscopic balloon probes, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0075574 discloses devices for vulnerable plaque detection that utilize optical fiber temperature sensors, and is incorporated by reference herein in its entirety.

U.S. Publication No. 2005/0165315 discloses a side-firing fiber-optic array probe, and is incorporated by reference herein in its entirety.

U.S. Publication No. 2006/0139633 discloses the use of high wavenumber Raman spectroscopy for evaluating tissue, and is incorporated by reference herein in its entirety.

The basket-style catheters known in the art have limitations due to the larger than optimal profile they present in blood vessels, the complexity of the mechanisms used to control radial expansion of the basket sections of the catheters (such as pull wire mechanisms) and the resulting loss of contact or close proximity of the basket arms with the vessel wall.

### SUMMARY OF INVENTION

One embodiment of the invention provides a balloon-expandable intravascular optical catheter that includes:

an elongate body having a proximal end and a distal end;

a distal basket section comprising a plurality of radially extendable, flexible probe arms and having a proximal end and a distal end wherein each probe arm comprises at least one optical fiber entering the probe arm and terminating at or near the most radially extendable portion of the probe arm in a side-viewing configuration or assembly to form a side-viewing optical probe element capable of transmitting and collecting light;

a guidewire tube radially centrally disposed along the body of the catheter and passing centrally through the basket section of the catheter, the distal end of the guidewire tube that includes:
retainers disposed circumferentially about the outer surface of the guidewire tube, each sized and configured to slideably retain the distal end of one of a probe arm, and
a distal cap segment sized and configured to shroud the retainers and to permit the distal ends of the probe arms to longitudinally slide within the distal cap segment,

the distal end of each probe arm slideably retained by a retainer and the distal end of each probe arm sized to prevent it from passing in a proximal direction through the retainer with which it is slideably retained; and

an expansion balloon circumferentially surrounding the guidewire tube within the basket section of the catheter and surrounded by the probe arms of the basket section, said expansion balloon comprising a lumen in fluid contact with at least one fluid channel running to the proximal end of the body of the catheter and being sized and configured to cause radial extension of the probe arms by inflation of the expansion balloon. The probe arms are flexible and return to a radially non-extended state when the balloon is deflated.

In one variation, each probe arm includes or consists essentially of a polymer tube in which the at least one optical fiber of the probe arm is disposed. In another variation, each probe arm includes or consists essentially of: a polymer tube in which the at least one optical fiber of the probe arm is disposed; and a metallic support finger disposed within the tube, wherein the metallic support finger is radially disposed more centrally than the at least optical fiber in order to permit radial viewing by the at least one optical fiber. A unitary metallic support structure may present each of the metallic fingers.

In still another variation, each probe arm includes or consists essentially of: an at least flat, elongate finger and at least one optical fiber, wherein the finger is radially disposed more centrally than the at least optical fiber in order to permit radial viewing by the at least one optical fiber, said finger and at least one optical fiber collectively encased in a polymeric encasement. The polymeric encasement material may, for example, be CTFE homopolymer, or another fluoropolymer that lacks C—H bonds. The basket section may include a unitary metallic support structure having an at least substantially tubular proximal portion from which each of the metallic fingers distally extends.

In any of the embodiments and variations, the polymeric encasement consists essentially of heat shrunken, heat shrink tubing.

The distal cap segment may provide a centrally disposed aperture by which a guidewire passing through the guidewire tube can exit the distal end of the catheter.

The majority of the expansion balloon may be disposed with in the proximal half of the basket section. For example, essentially all of the expansion balloon in its unexpanded state may be disposed within the proximal half of the basket section. The catheter may sized and configured for intravascular interrogation of a blood vessel wall, such as a human coronary artery or carotid artery.

In one variation, the polymeric material enclosing or encasing the at least one optical fibers of each probe arm: has an least substantially non-discernable Raman scattering signal in one or more preselected wavenumber regions used for analysis of a target; is adequately transparent to excitation light delivered via the at least one optical fibers to illuminate a target; and is adequately transparent to Raman-scattered light in the preselected wavenumber range that is collected from the illuminated target. The preselected wave number range may, for example, be or be within the Raman high wavenumber region.

The at least two flexible probe arms may, for example, consist of six to eight circumferentially spaced, flexible probe arms.

A related embodiment of the invention provides an optical intravascular catheter system/apparatus that includes:
an optical catheter according to the invention as described herein;
a light source in optical communication with the optical probe elements of the probe arm(s) and suitable for generating Raman spectra; and
a Raman spectrometer in optical communication with the optical probe elements of the probe arm(s).

In one variation, the system/apparatus further includes at least one computer that includes at least one processor and computer accessible memory. The computer receives, stores and/or analyzes data collected by the system and/or controls the system. The computer may be operably linked to one or more user input devices such as a keyboard and one or more output devices such as a display and a printer.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIGS. 1A-C show an embodiment of the invention in which optical fibers (104) are disposed in probe arms formed of polymer tubing (103). FIG. 1D exemplifies one aspect of the embodiment.

FIGS. 2A-C show an embodiment of the invention in which optical fibers (104) are disposed in probe arms formed of polymer tubing (103) and further including a support member including finger members extending into each probe arm.

FIGS. 3A-C show an embodiment of the invention in which the probe arms of the basket section of the catheter are formed from a support member presenting a finger extending the length of each probe arm, an optical fiber extending distally into the probe arm and terminating at a viewing portion of the probe arm, wherein the optical fiber is located on the radially distal side of the finger, and a polymer encasement of the finger and optical fiber, such as heat-shrunken, heat shrink polymer tubing.

### DETAILED DESCRIPTION

The invention provides selectively balloon expandable basket-style catheter optical probes including probe arms having individually free-floating distal ends or collectively free-floating distal ends. The basket segment of the probe has a lumen that accommodates a guidewire through its length and includes a proximal end that remains static with respect to the catheter to which it is attached and a distal end segment that slideably surrounds the guidewire. Positioned between, and attached, to the each of the proximal and distal ends are probe arms that have an outward radial bias. The slideable distal end(s) of the basket segment permits radial expansion and contraction of the probe arms by way of changes in the distance between the proximal and distal ends of the probe as the probe travels within a lumen so that close contact or close proximity of the individual probe arms to the vessel wall is maintained even as the basket section of the catheter traverses tortuous bends. An expansion control balloon having a lumen is positioned within the basket section (underlying the probe arms), circumferentially surrounding a centrally disposed guide wire tube or tubing segment. The lumen of the expansion control balloon is in fluid communication with at least one channel leading down to the proximal end of the catheter through which fluid (liquid or gas) pressure may be applied and regulated to expand and deflate the balloon, thereby respectively radially expanding and radially collapsing the basket section of the catheter. The probe arms may have spring properties so that they radially extend only when radially outward force is applied by the expansion balloon and return to an unextended position when the expansion force is removed.

The basket-style probe assemblies of the invention provide for the delivery and/or collection of diagnostic and/or therapeutic energy such as light in small spaces. The probe assemblies can be small and flexible and are well-suited to performing minimally invasive diagnostic examinations of biological tissues in vivo. Preferred embodiments of the invention are directed to configurations in which the probe arms are constructed in a manner to simplify and reduce the overall profile of the catheter. Prior art, mechanically deployable basket catheter are characterized by a suboptimally large diameter due to the pull-wire design, the risk of pull-wire detachment and the tendency of the pull-wire based mechanisms to cause the catheter tip to pull to one side. Also, the pull-wire designs cause snaking in the shaft of a pull wire deployed basket. In addition, fixed distal basket designs (those in which the distal end of the probe arms are not free-floating, either together or independently) that were tested caused the basket to prolapse when traversing a curve and are also difficult to manufacture. In contrast, with the present invention, the combination of the balloon expansion mechanism, free-floating distal segment(s) of the basket and improved probe arm configurations provides a smaller diameter profile with superior control, agility and optical interrogation of the vessel wall versus prior art intravascular basket catheter designs, unhindered by the aforementioned shortcomings of the prior designs.

In one embodiment of the invention, the distal end of each probe arm is independently, freely slideable in a distal end cap of the cather to automatically adjust the proximal to distal length of the probe are, such as in the manner of U.S. Publication No. 2004/0176699 A1, which is incorporated by reference herein in its entirety. In another embodiment the distal ends of the probe arms are fixably joined to a distal end segment of the catheter which freely (slideably) surrounds a guidewire, as in the manner of U.S. Publication No. 2008/0045842 A1 which is incorporated by reference herein in its entirety.

The present invention utilizes hydraulic pressure to expand a small balloon to expand the probe arms symmetrically to be in close proximity to or in contact with the vessel wall. The size of the balloon may also be selected to allow blood flow to continue through the vessel whilst the basket is radially deployed. The tip of the catheter does not pull to one side when the basket section is radially deployed because the balloon is centered in the catheter and because the distal end of the array is not fixed allowing the free movement of each arm. The distal portion of the probe arms not being fixed allows each of the free ends of the arms to slide independently within an end cap which is attached to the distal portion of the inner member. When traversing a curve, this allows the arms on the inside of the curve to push towards the distal end of the end cap while the arms on the outside of the curve will pull toward the proximal end of the end cap allowing the arms to maintain circumferential position within the vessel. Because hydraulic pressure is used to expand the balloon, thereby radially deploying the probe arms, rather than pulling on a wire, the pull-wire detachment issue no longer exists.

Pull-wire basket catheters also exert more pressure on the vessel wall unless perfectly expanded to the vessel diameter which, in practical terms is not obtainable, whereas the balloon deployed basket catheter embodiment of the invention with independently adjusting (individually free-floating) probe arms self-limit the pressure exerted on the vessel wall since by way of compensatory, automatic adjustment of the probe arms by movement into the distal cap of the catheter.

The invention is further described below with reference to the appended figures.

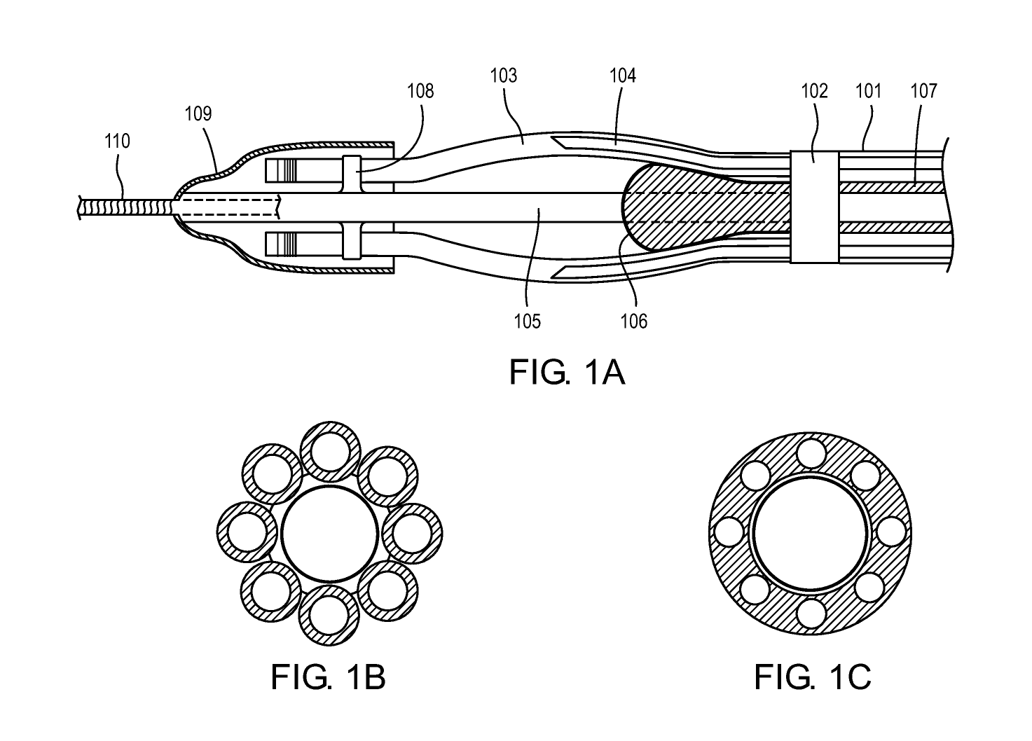

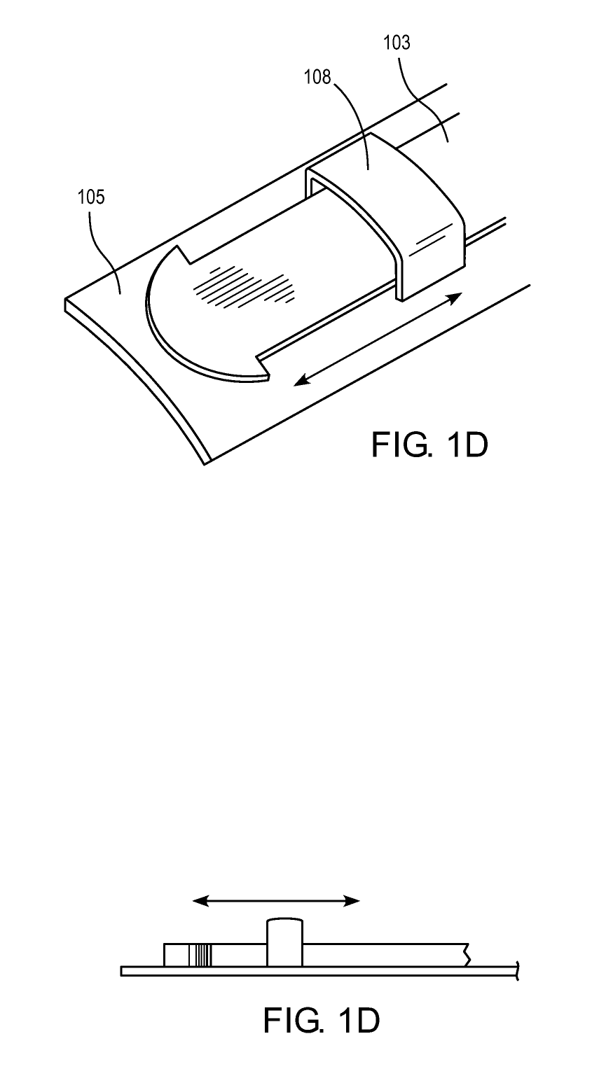

FIGS. 1A-C show a balloon-expandable catheter embodiment of the invention in which optical fibers (104) are disposed in probe arms formed of polymer tubing (103). The catheter has an elongate proximal body 101 disposed proximally to the basket section of the catheter. An annular collar member 102 hold the probe arms 103 (and others) in a circumferentially separated configuration. The probe arms are formed from a polymeric tube in which one or more side-viewing optical fibers 104 extend to a viewing portion of the probe arm, i.e., the part of the probe arm that will most radially extend when the basket section of the catheter is radially extended. Optical fiber 104 runs back to the proximal end of the catheter where it is connected to one or more of a light source and a light detector or analyzer such as a spectrometer. The catheter further includes a radially centrally disposed guidewire tube that extends the full length of the catheter. Surrounding at least part of guidewire tube 105 in the basket section of the catheter is an expansion balloon 106 the inflation and deflation of which is used to control the radial expansion and contraction of the probe arms of the basket section. One or more fluid channels 107 in fluid communication with the lumen of balloon 106 run down to the proximal end of the catheter to a pressure controller. Channel 107 may, for example, be an annular channel that surrounds guidewire tube 105. Fixably connected to guidewire 105 tube are retainers 108 that slideably retain the distal ends of probe arms 103. The distal ends of probe arms 103 are widened so that they cannot pass through retainers 108 (as exemplified schematically in FIG. 1D). The widening of the distal end of the probe arm may be achieved in any manner, for example, by inserting a widened plug member into the distal end of the tube forming the probe arm or by molding/pressing the distal end of the tube under heat so that it widens. A distal cap member 109 is fixably connected to the distal end of guidewire tube 105 and shrouds retainers 108 and the distal ends of prober arms 103. A guidewire 110 is seen extending out of an aperature in distal cap member 109 and thus out of guidewire tube 105. FIG. 1B shows a cross-sectional, end-on view of the basket section of the catheter (not showing the optical fibers within the tubes of the probe arms). FIG. 1C shows a cross-section of the catheter within collar member 102.

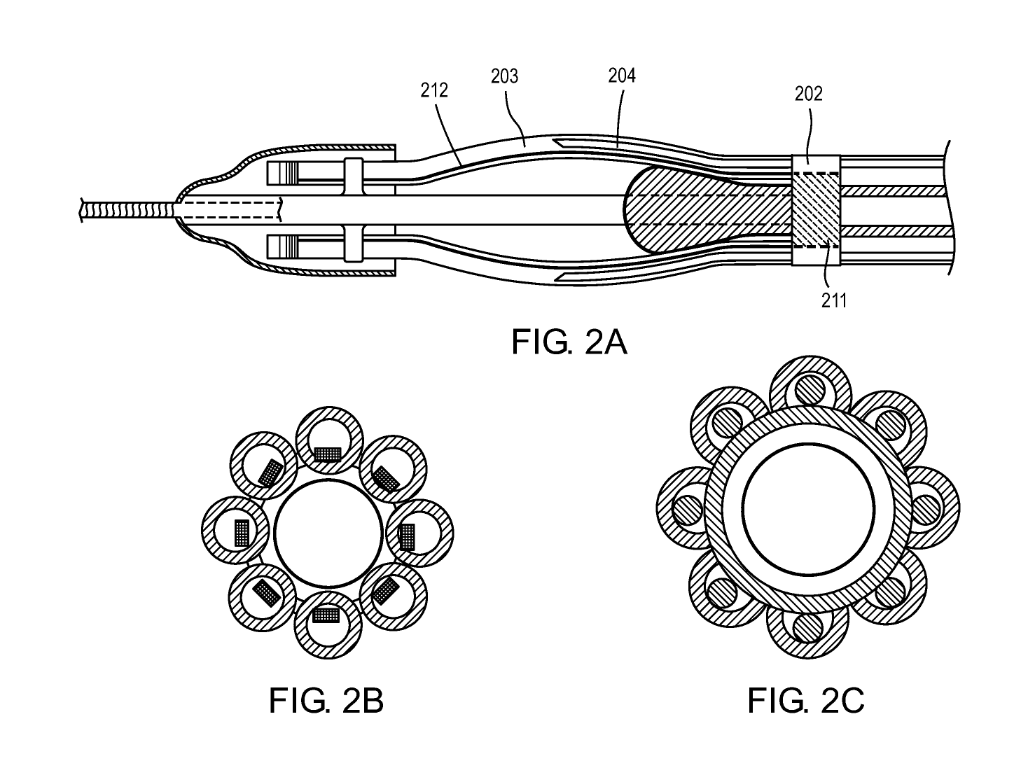

FIGS. 2A-C show an embodiment of the invention in which optical fibers 204 are disposed in probe arms formed of polymer tubing 103 and further including a support member having a tubular portion 211 including finger members 212 extending into each probe arm. The support structure may, for example, be formed from a metallic tube. The fingers of the support member may be at least substantially flat and rectangular in cross-section. Optical fibers 204 are located on the radially more distal side of fingers 212 so that the optical fibers can deliver and receive light through the tubing of the probe arm unobstructed by the fingers.

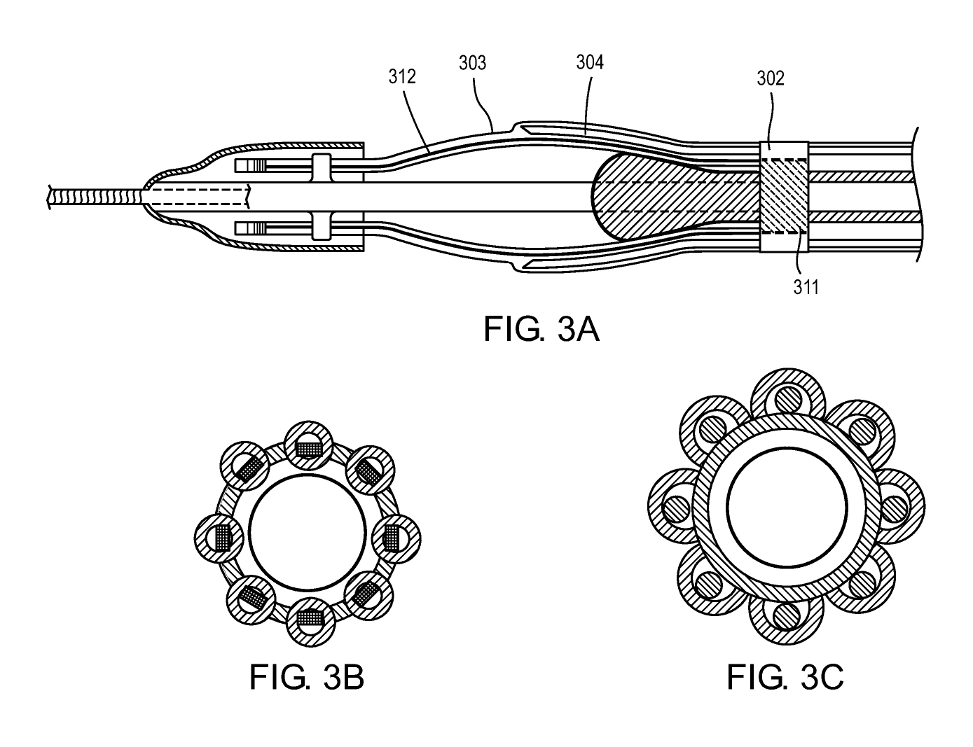

FIGS. 3A-C show an embodiment of the invention in which the probe arms of the basket section of the catheter are formed from a support member presenting a finger extending the length of each probe arm, an optical fiber 304 extending distally into the probe arm and terminating at a viewing portion of the probe arm, wherein the optical fiber is located on the radially distal side of the finger, and a polymer encasement 303 of the finger and optical fiber, such as heat-shrunken, heat shrink polymer tubing. Such a configuration simplifies manufacturing of the probe arms and advantageously further reduces the profile of the probe arms.

As referred to herein, the term “probe arm” means one of the flexible elements that is disposed between the proximal end and distal end of the basket section and which contacts or nears a lumen wall, such as an artery wall, by radial expansion. One or more of the probe arms may include an operable probe element or sensor, also referred to as a scanning core herein, for delivering and/or receiving diagnostic or therapeutic energy, for example, light, ultrasound or heat.

The figures show 2-probe arms for illustration. However, catheters of the invention may generally have at least two probe arms and may, for example, have 2, 3, 4, 5, 6, 7 or 8 probe arms. By using multiple circumferentially spaced probe arms, a composite radial field-of-view can be built up. The probe arms may be at least substantially uniformly circumferentially spaced. The 6-probe arm configuration provides an excellent balance of radial coverage for optical interrogation and maneuverability, in a catheter sized for interrogation of human, e.g., adult, coronary arteries.

The particular configuration shown in the accompanying figures is an “over the wire” catheter with a guidewire lumen passing the entire length of the catheter, and out through the “guidewire port” on the hub. For simplicity, the remaining descriptions will discuss the optical spectroscopy catheter embodiments, but the invention is not limited to this modality and may, for example, be additionally or alternatively implemented with other diagnostic modalities such as ultrasound (IVUS), MRI, OCT or thermography.

The optical fiber bundles may begin within each distal scanning optic core and extend to proximal to connectors which interface with a light source and detector. Each optical fiber bundle may contain one or more optical fibers.

For optical probe elements, a lateral field-of-view may be provided by any suitable means, for example, by using a mirror or prism in optical communication with the one or more optical fibers and/or by using angle-cut optical fiber faces. For example, a 45-degree mirror or prism may be used to laterally redirect light with respect to a distal scanning core of a probe.

The probe arms may be provided with an outward radial shape or “bias” of the for tissue contact or proximity may, for example, by utilizing probe arms with a pre-set curvature. The bias helps to ensure that the viewing portion of the probe arms is the part that extends most radially to contact or near a vessel wall. However, the probe arms do not “self-extend.” It is radially outward force applied by the expansion balloon that radially extends the probe arms. The probe arms may be formed from plastic/polymer tubing or segments having a curvature that provides the outward radial shape for tissue contact. Another approach is to provide this support via an internal structural element. The support/reinforcement member may be a unitary structure, i.e., a one-piece structure. The support structure may be constructed from a tube may, for example, be made from a stainless steel, a spring steel, superelastic Nitinol alloy or a polymeric material such as CTFE homopolymer, PEEK, Polyimide, Polyamide, PTFE or other engineering materials for medical device construction, wherein one end (the proximal end) remains a tubular segment from which the individual probe arms distally extend. The basket reinforcement element tube may, for example be fabricated in a collapsed form (laser cut thin-walled tubing) and then compressed (with respect to its lateral axis) within a mold base and heat treated to set the preferred unconstrained shape. Injection molding or thermoforming of plastic/polymer materials may also be used to fabricate the basket reinforcement element.

The invention also provides a method for diagnostically interrogating and/or treating a body lumen wall, such as blood vessel lumen wall, that includes the steps: of inserting a catheter according to the invention into a body lumen, such as a blood vessel lumen; and delivering diagnostic and/or therapeutic energy via at least one probe element on at least one probe arm of the catheter to the lumen wall. The energy may for example, be light energy. Energy received via the probe elements or measured by the probe elements may be analyzed to evaluate and diagnose a subject tissue. The invention is not limited by the method used to interrogate and diagnosis the condition of a blood vessel wall. Optical and/or non-optical methods may be used. Multiple methods may also be used. Suitable optical methods include, but are not limited to, low-resolution and high resolution Raman spectroscopy, fluorescence spectroscopy, such as time-resolved laser-induced fluorescence spectroscopy, and laser speckle spectroscopy. Photoacoustic stimulation in conjunction with acoustical detection by any means may also be used. One embodiment of the invention is a method for diagnosing and/or locating one or more atherosclerotic lesions, such as vulnerable plaque lesions, in a blood vessel, such as a coronary artery of a subject, using a catheter as described herein to evaluate the properties of a vessel wall, such an artery, at one more locations along the vessel. In any of the embodiments, the catheter including its basket section and probe arms thereof may be sized for interrogation of human coronary arteries.

Differentially diagnosing, identifying and/or determining the location of an atherosclerotic plaque, such as a vulnerable plaque, in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differentials characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Time-resolved laser-induced fluorescence methods for characterizing atherosclerotic lesions are disclosed in U.S. Pat. No. 6,272,376, which is incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated by reference herein in its entirety. A method and system for detecting and localizing vulnerable plaques based on the detection of biomarkers is disclosed in U.S. Pat. No. 6,860,851, which is hereby incorporated by reference herein in its entirety.

Raman spectroscopy has proven capable of determining the chemical composition of tissues and diagnosing human atherosclerotic plaques. Typical methods of collecting Raman scattered light from the surfaces of artery do not register information about how far the scattering element is from the collection optics. Two wavenumber regions that yield useful information for evaluating the condition of blood vessels are the so-called Raman fingerprint region i.e., approximately 200 to 2,000 cm⁻¹, and the so-called high wavenumber region, i.e., approximately 2,600 to 3,200 cm⁻¹. The collection of Raman spectra in the fingerprint (FP) region, through optical fibers is complicated by Raman “background” signal from the fibers themselves. In order to collect uncorrupted FP spectra, complicated optics and filters on the tips of catheters and often these designs require the use of multiple fibers. Since the Raman scattered signal is weak, large multimode fibers are utilized in the multi-fiber catheter designs, which creates an unwieldy catheter that is less than optimal for exploring delicate arteries, such as the human coronary arteries. However, common optical fiber materials generate very little Raman background signal in the high wavenumber region, permitting a simplified, single optical fiber probe element implementation of intravascular Raman spectroscopy.

Since cholesterol and its esters have distinctive Raman scattering profiles within the Raman high wavenumber region, the use of the Raman high wavenumber region for analysis is particularly useful for locating and characterizing lipid-rich deposits or lesions as may occur in blood vessels, such a vulnerable plaques in arteries, such as the coronary arteries.

One embodiment of the invention provides a method for evaluating the wall of a blood vessel such an artery, such as a coronary artery, such as a human coronary artery, that includes the steps of:
providing any of the intravascular basket catheter embodiments of the invention having a proximal end and a distal insertion end including a basket section comprising at least two radially extendable wall-contacting optical probe arms;
disposing the basket section of the catheter in a blood vessel; and
taking optical readings of the vessel wall at one or more locations in the blood vessel using the at least two optical probe arms.

In one variation, the method includes transmitting light, such as laser light, from a light source to target regions of a lumen wall, such as a blood vessel wall, via the scanning core of the probe arms of the catheter and collecting and analyzing inelastically scattered (Raman scattered) light resulting from the illumination of the target regions using a Raman spectrometer. The Raman spectrometer may be configured to measure Raman scattered light in the high wavenumber region and/or the fingerprint region and the data for either or both of the regions may be analyzed to determine the chemical composition of the target regions and/or diagnose the target regions/tissue.

In another variation, the method includes transmitting light, such as laser light, for fluorescence stimulation of the target regions of a lumen wall, such as a blood vessel wall, via the scanning core of the probe arms of the catheter and collecting and analyzing fluorescent emissions resulting from the illumination of the target regions using a fluorescence spectrometer. In a sub-variation, time-resolved laser-induce fluorescence is performed using a catheter embodiment of the invention.

It should be understood for the above methods that the probe arms are radially extended to contact or closely near the vessel walls in order to take readings from the probe arm-disposed optical assemblies. The step of taking readings may include taking the recited readings at more than one longitudinal location in a blood vessel, for example, while the catheter is pulled back by operation of a catheter pullback mechanism.

The invention also provides an integrated system for evaluating the status of a lumen wall such as a blood vessel wall, for example, for diagnosing and/or locating vulnerable plaque lesions, that includes a basket-style catheter according to the invention having probe elements (scanning cores) for interrogating the lumen wall that are in communication with an analyzer for analyzing the signal and/or information received via the probe elements. The analyzer may include a computer.

A related embodiment provides an integrated system for optically evaluating the status of a lumen wall, such as a blood vessel wall, for example, for diagnosing and/or locating atherosclerotic lesions, such as vulnerable plaque lesions in an artery, that includes an basket-style catheter according to the invention having optical probe elements for interrogating the blood vessel in communication with a light source such as a laser for illuminating a target region of a blood vessel via the catheter and a light analyzer, such as a spectrometer, for analyzing the properties of light received from the target region via the catheter.

A related embodiment of the invention provides a diagnostic catheter system for the evaluation of blood vessel walls that includes an intravascular diagnostic catheter as described herein, a light source such as a laser for stimulating Raman scattered light emissions from a target region via the wall-contacting portion (scanning core) of the probe arms of the catheter, and a Raman spectrometer for analyzing Raman scattered light collected from a target via the wall-contacting portion of the probe arms of the catheter. The system may be configured to collect and analyze Raman spectral data within the region of approximately 2,600 to 3,200 cm⁻¹, i.e., the so-called high wavenumber region, and/or the within the region of approximately 200 to 2,000 cm⁻¹, i.e., the so-called fingerprint region. The optical probe arms may, for example, each have a single optical fiber and the system may be configured to perform high wavenumber Raman spectroscopy from each probe arm via the single optical fiber.

One or more computers, or computer processors generally working in conjunction with computer accessible memory, may be part of any of the systems for controlling the components of the system and/or for analyzing information obtained by the system.

Co-owned U.S. Publication No. 2008/0129993 A1 (application Ser. No. 11/876,899), which is incorporated by reference in its entirety, teaches windowless optical probe assemblies for use with Raman spectroscopy, such as high wavenumber Raman spectroscopy, which may be implemented with the present invention. In accordance with this application, the probe arms may include one or more optical fibers housed in material(s) having a very low Raman scattering cross-section in the wavenumber region used for analysis of a target and being adequately transparent to excitation light delivered via the fiber optics to the target and to Raman-scattered light (inelastically scattered light) collected from the irradiated target in the desired wavenumber range. In this manner, a separate window or aperture is not needed in the viewing portion (scanning core region) of the probe arm to permit illumination and collection of light having the desired ranges of wavelengths, thereby simplifying manufacture and improving performance of the catheter.

Thus, in any of the embodiment of the present invention, at least one of the probe arms may include: an optical fiber assembly having a viewing portion (scanning core portion) for transmitting and receiving light, wherein at least the viewing portion of the optical fiber assembly is enclosed in a material, such as a polymeric material, having an at least substantially non-discernable Raman scattering signal (a level not interfering with analysis) in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly. The optical fiber assemblies of the probes and catheter probes may include one or more optical fibers. In one variation, the main bodies of the probe arms (excluding the optical fiber assemblies) may be entirely composed of or enclosed in the polymeric material. The preselected wavenumber region may, for example, be in the range of approximately 2,600 to 3,200 cm⁻¹, i.e., within the high wavenumber region. For the high wavenumber region, the enclosure material may, for example, include or consist of polymer material that at least substantially does not include carbon-hydrogen bonds, such as polytetrafluoroethylene (PTFE), fluorinated ethylene-propylene (FEP) and perfluoroalkoxy polymer resin (PFA). In these cases, the excitation wavelength used to obtain the high wavenumber spectra may, for example, be at or around 740 nm, or at a suitable near infrared wavelength generally. The light source may be a laser, such as a single-mode laser or a wavelength stabilized multi-mode laser diode, such as a Volume Bragg Grating Stabilized multi-mode laser diode (available, e.g., from PD-LD, Inc., Pennington, N.J.)

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A balloon-expandable intravascular optical catheter, comprising:
an elongate body having a proximal end and a distal end;
a distal basket section comprising a plurality of radially extendable, flexible probe arms and having a proximal end and a distal end wherein each probe arm comprises at least one optical fiber entering the probe arm and terminating at or near the most radially extendable portion of the probe arm in a side-viewing configuration or assembly to form a side-viewing optical probe element capable of transmitting and collecting light;
a guidewire tube radially centrally disposed along the body of the catheter and passing centrally through the basket section of the catheter, the distal end of the guidewire tube comprising:
retainers disposed circumferentially about the outer surface of the guidewire tube, each sized and configured to slideably retain the distal end of one of a probe arm, and
a distal cap segment sized and configured to shroud the retainers and to permit the distal ends of the probe arms to longitudinally slide within the distal cap segment,
the distal end of each probe arm slideably retained by a retainer and
the distal end of each probe arm sized to prevent it from passing in a proximal direction through the retainer in which it is slideably retained; and
an expansion balloon circumferentially surrounding the guidewire tube within the basket section of the catheter and surrounded by the probe arms of the basket section, said expansion balloon comprising a lumen in fluid contact with at least one fluid channel running to the proximal end of the body of the catheter and being sized and configured to cause radial extension the probe arms by inflation of the expansion balloon.

2. The catheter of claim 1, wherein each probe arm comprises:
a polymer tube in which the at least one optical fiber of the probe arm is disposed.

3. The catheter of claim 1, wherein each probe arm comprises of:
a polymer tube in which the at least one optical fiber of the probe arm is disposed; and a metallic support finger disposed within the tube,
wherein the metallic support finger is radially disposed more centrally than the at least optical fiber in order to permit radial viewing by the at least one optical fiber.

4. The catheter of claim 3, further comprising a unitart metallic support structure that presents each of the metallic fingers.

5. The catheter of claim 1, wherein each probe arm comprises:
an at least flat, elongate finger and at least one optical fiber, wherein the finger is radially disposed more centrally than the at least optical fiber in order to permit radial viewing by the at least one optical fiber,
said finger and at least one optical fiber collectively encased in a polymeric encasement.

6. The catheter of claim 5, wherein the basket section comprising a unitary metallic support structure having an at least substantially tubular proximal portion from which each of the metallic fingers distally extends.

7. The catheter of claim 5, wherein the fingers are metallic.

8. The catheter of claim 5, wherein the polymeric encasement consists essentially of heat shrunken, heat shrink tubing.

9. The catheter of claim 1, wherein the distal cap segment provides a centrally disposed aperture by which a guidewire passing through the guidewire tube can exit the distal end of the catheter.

10. The catheter of claim 1, wherein at least the majority of the expansion balloon is disposed with in the proximal half of the basket section.

11. The catheter of claim 10, wherein essentially all of the expansion balloon in its unexpanded state is disposed within the proximal half of the basket section.

12. The catheter of claim 1, wherein the catheter is sized and configured for intravascular interrogation of a blood vessel wall.

13. The catheter of claim 12, wherein the blood vessel wall is a human coronary artery wall.

14. The catheter of claim 2, wherein the polymeric material enclosing or encasing the at least one optical fibers of each probe arm:
has an least substantially non-discernable Raman scattering signal in one or more preselected wavenumber regions used for analysis of a target;
is adequately transparent to excitation light delivered via the at least one optical fibers to illuminate a target; and
is adequately transparent to Raman-scattered light in the preselected wavenumber range that is collected from the illuminated target.

15. The catheter of claim 14, wherein the preselected wave number range is or is within the Raman high wavenumber region.

16. The catheter of claim 1, wherein the at least two flexible probe arms consist of six to eight circumferentially spaced, flexible probe arms.

17. An optical intravascular catheter system, comprising:
an optical catheter according to any previous claim;
a light source in optical communication with the optical probe elements of the probe arm and suitable for generating Raman spectra; and
a Raman spectrometer in optical communication with the optical probe elements of the probe arm.

18. The system of claim 17, further comprising at least one computer processor.

19. The system of claim 17, wherein the preselected wave number range is or is within the high wavenumber region.
